(19) United States
(12) Patent Application Publication
(10) Pub. No.: US 2021/0163437 A1
(43) Pub. Date: Jun. 3, 2021

(54) DERIVATIVES OF TRICYCLIC SPIROLACTONES AND USES THEREOF IN TREATMENT AND MANAGEMENT OF PAIN

(71) Applicant: Yissum Research Development Company of The Hebrew University of Jerusalem Ltd., Jerusalem (IL)

(72) Inventors: Dmitry TSVELIKHOVSKY, Nof Hagalil (IL); Avi PRIEL, Tel Aviv (IL)

(73) Assignee: Yissum Research Development Company of The Hebrew University of Jerusalem Ltd., Jerusalem (IL)

(21) Appl. No.: 16/613,564

(22) PCT Filed: May 17, 2018
(86) PCT No.: PCT/IL2018/050546

§ 371 Date: Nov. 14, 2019

Related U.S. Application Data
(60) Provisional application No. 62/507,394, filed on May 17, 2017.

(51) Int. Cl. C07D 307/93; C07D 307/83

(52) U.S. Cl. CPC C07D 307/93; C07D 307/83

(57) ABSTRACT

The present application relates to derivatives of tricyclic spirolactones compounds of formula (I) and compositions thereof and uses thereof in treatment and management of pain. Formula (I).

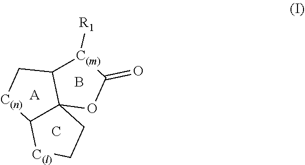

### BACKGROUND OF THE INVENTION

TRPV1 is a prominent signal integrator of the pain system, known to be activated by vanilloids, a family of endogenous and exogenous pain-evoking molecules, through the vanilloid-binding site (VBS). The extensive preclinical profiling of small molecule inhibitors provides intriguing evidence that TRPV1 inhibition can be a useful therapeutic approach. However, the dissimilarity of chemical species that activate TRPV1 creates a major obstacle to understanding the molecular mechanism of pain induction, which is viewed as a pivotal trait of the somatosensory system.

The Transient Receptor Potential Vanilloid-1 (TRPV1), also known as the “heat and capsaicin receptor”, is a prominent signal integrator of the somatosensory system.1,2 It is expressed primarily on pain fibers (Ad -and C-fibers) and has the unique ability to detect an array of noxious stimuli, including heat (≥42 ° C.), protons, polyunsaturated fatty acids (PUFAs), peptide toxins, and plant toxins. The TRPV1 expression pattern and its polymodality make it an intriguing target for pain treatment.

Vanilloids, both endogenous and exogenous, are pain-evoking molecules that activate TRPV1 through the vanilloid-binding site (VBS) located in the intracellular domain between the S3 and S4 transmembrane segments. This large and diverse family of TRPV1 ligands includes endovanilloids, such as the endocannabinoids anandamide, N-arachidonoyl dopamine (NADA), and lipoxygenase products of arachidonic acid. However, the most well-known and studied activators are the exo-vanilloid phytotoxins capsaicin and resiniferatoxin (RTX). The extensive preclinical profiling of small molecule TRPV1 inhibitors acting through the VBS provides intriguing evidence that TRPV1 inhibition can be a useful therapeutic approach for inflammatory, cancer, and neuropathic pain. Discerning the mechanism by which the TRPV1 VBS accommodates its abundance of diverse ligands would have important implications for future drug development efforts. A major obstacle to this endeavor is the inability to discern a lead motif within the diverse and chemically dissimilar range of known activating agents. Additionally, it is unclear which functional domains (out of the many characterized to date) are essential to elicit the desirable engagement with this receptor (i.e., activation/sensitization).

To get a further understanding the inventors have analyzed known agonists and antagonists (selective and non-selective, specific and non-specific, natural and synthetic compounds) of TRPV1 which were categorized into six distinctive families, each displaying unique chemical and functional characteristics. FIG. 1 illustrates representative compounds for these groups. As observed from the chemical architectures of naturalendogenous and exogenous agonists (FIG. 1A), there are several common molecular domains.

Numerous structure-activity relationship (SAR) studies reveal three similar, yet distinct regions: 1) the vanilloid scaffold, 2) the carbonylic site (ester or amide), and 3) the lipophilic domain.

Previous studies have demonstrated that the vanilloid segment is important for the biological activity of agonists. In the absence of this unit, all the compounds studied were shown to be inactive. The carbonylic segment is integrated through a simple and inactivated ester or amide and is responsible for specific hydrogen bond interactions between the substrate and the receptor. The lipophilic aliphatic domain is another common region that is significantly different among agonists but is crucial for potency.

While examining the vast variety of TRPV1 antagonists, it was clear to the inventors that no common functional denominator (a requisite chemical/structural motif) can be identified among this spectrum of compounds. Some of the compounds slightly resemble one another, but others are completely unique (FIG. 1B). Despite having cardinally different pharmacophores and functional domains, it is safe to assert that all known TRPV1 antagonists consist of three essential interactional features: a hydrogen-bond acceptor, a hydrogen-bond donor, and a ring feature (in most cases aromatic).

To date, TRPV1 activation/inactivation through the VBS (either by agonists, or antagonists) has relied on the presence of these functional/structural domains. The majority of recent research efforts in this field have examined the interaction between an operational pharmacophore and the TRPV1-VBS binding site. However, conventional drug discovery efforts regard the presence of one of these groups as an indispensable requirement for any potential therapeutic agent/drug candidate.

The inventors have now shown the existence of a conceptually different family of TRPV1-activating agents acting through the VBS. This series of molecules contains none of the structural domains previously believed to be crucial for triggering the receptor-agent interactions. These compounds are highly compact, tricyclic, spherical spirolactones that share an angularly fused topology.

### SUMMARY OF THE INVENTION

Here, we establish the existence of a unique synthetic agonists that interface with TRPV1 through the VBS, containing none of the molecular domains previously believed to be required for this interaction. The overarching value obtained from our inquiry is the novel advancement of the existing TRPV1 activation model. These findings uncover new potential in the area of pain treatment, providing a novel synthetic platform.

Thus, the present invention provides a compound of general formula (I):

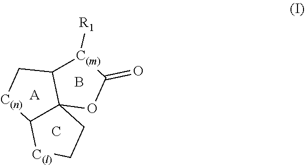

Wherein R₁ is selected from straight or branched C₁-C₁₀ alkyl, straight or branched C₂-C₁₀ alkenyl, straight or branched C₂-C₁₀ alkynyl; each optionally interrupted by at least one heteroatom and optionally substituted by at least one aryl group optionally substituted with at least one of —CN, —OH, alkoxy, amine, amide and halogen;

Rings A, B and C are each optionally a saturated or unsaturated ring having optionally at least one heteroatom; and are each optionally substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;

R₄, R₅, R₆, R₇ are each independently selected from H, halogen, —OH, —NH₂, straight or branched C₁-C₁₀ alkyl, straight or branched C₂-C₁₀ alkenyl, straight or branched C₂-C₁₀ alkynyl, aryl, —O(C ₁-C₁₀)alkyl, NH₂, amine; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;

n is an integer selected from 1-10; —C₍ₙ₎— is selected from a straight or branched alkylene, straight or branched alkenylene, straight or branched alkynylene; optionally interrupted by at least one heteroatom;

m is an integer selected from 1-10; —C₍ₘ₎— is selected from a straight or branched alkylene, straight or branched alkenylene, straight or branched alkynylene; optionally interrupted by at least one heteroatom;

l is an integer selected from 1-10; —C₍ₗ₎— is selected from a straight or branched alkylene, straight or branched alkenylene, straight or branched alkynylene; optionally interrupted by at least one heteroatom.

In another one of its aspects, the invention provides a compound of general formula (II):

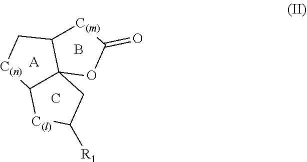

Wherein R₁ is selected from straight or branched C₁-C₁₀ alkyl, straight or branched C₂-C₁₀ alkenyl, straight or branched C₂-C₁₀ alkynyl; each optionally interrupted by at least one heteroatom and optionally substituted by at least one aryl group optionally substituted with at least one of —CN, —OH, alkoxy, amine, amide, halogen;

Rings A, B and C are each optionally a saturated or unsaturated ring having optionally at least one heteroatom; and are each optionally substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;

R₄, R₅, R₆, R₇ are each independently selected from H, halogen, —OH, —NH₂, straight or branched C₁-C₁₀ alkyl, straight or branched C₂-C₁₀ alkenyl, straight or branched C₂-C₁₀ alkynyl, aryl, —O(C₁-C₁₀)alkyl, NH₂, amine; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;

n is an integer selected from 1-10; —C₍ₙ₎— is selected from a straight or branched alkylene, straight or branched alkenylene, straight or branched alkynylene; optionally interrupted by at least one heteroatom;

m is an integer selected from 1-10; —C₍ₘ₎— is selected from a straight or branched alkylene, straight or branched alkenylene, straight or branched alkynylene; optionally interrupted by at least one heteroatom;

l is an integer selected from 1-10; —C₍ₗ₎— is selected from a straight or branched alkylene, straight or branched alkenylene, straight or branched alkynylene; optionally interrupted by at least one heteroatom.

In some embodiments, R₁ is straight or branched C₁-C₁₀ alkyl. In some embodiments, R₁ is straight or branched C₂-C₁₀ alkenyl. In other embodiments, R₁ is selected from straight or branched C₁-C₁₀ alkyl, straight or branched C₂-C₁₀ alkenyl that is interrupted by at least one heteroatom. In further embodiments, R₁ is selected from straight or branched C₁-C₁₀ alkyl, straight or branched C₂-C₁₀ alkenyl substituted by at least one phenyl optionally substituted with at least one of —CN, —OH, alkoxy, amine, amide and halogen. In other embodiments, R₁ is selected from straight or branched C₁-C₁₀ alkyl, straight or branched C₂-C₁₀ alkenyl substituted by at least one phenyl substituted with at least one of —CN, —OH, alkoxy, amine, amide and halogen.

In some embodiments, wherein Ring C is a saturated ring. In other embodiments, Ring C is a saturated ring substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl.

In further embodiments, Ring C is an unsaturated ring. In other embodiments, Ring C is an unsaturated ring substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl.

In some embodiments, m=1, —C(m)- being a C1-alkylene optionally interrupted by at least one heteroatom. In some embodiments, n=1, —C(n)- being C1-alkylene, optionally interrupted by at least one heteroatom. In further embodiments, n=2, —C(n)- being C2-alkylene, optionally interrupted by at least one heteroatom. In other embodiments, n=3, —C(n)- being C3-alkylene, optionally interrupted by at least one heteroatom. In further embodiments, l=1, —C(l)- is a C1-alkylene, optionally interrupted by at least one heteroatom. In further embodiments, l=2, —C(l)- is a C2-alkenylene, optionally interrupted by at least one heteroatom.

In some embodiments, a compound of the invention is selected from:

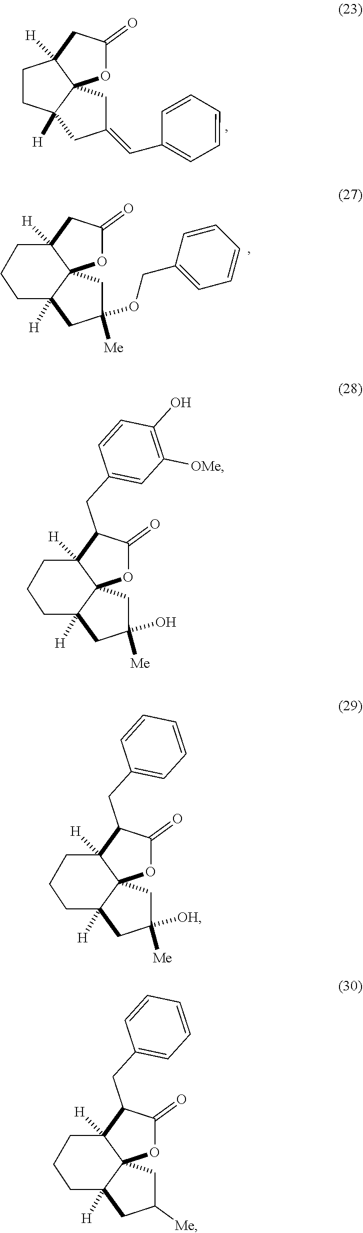

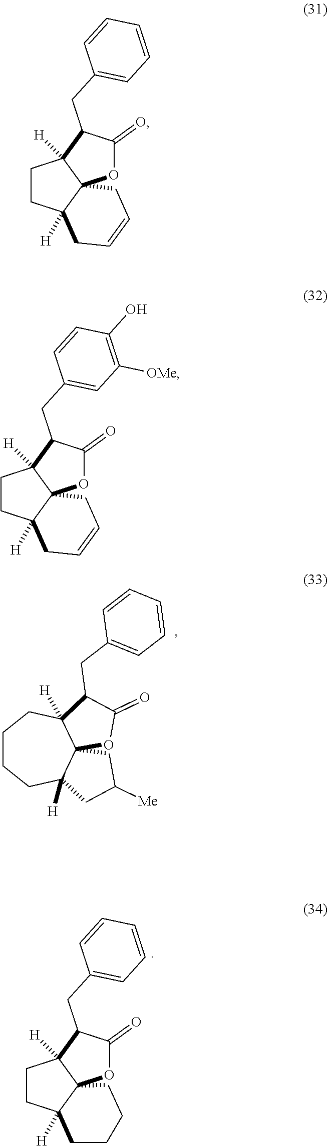

The invention further provides a pharmaceutical composition comprising at least one compound as defined hereinabove and below.

In another aspect, the invention provides a compound as defined herein above and below for use in the treatment of pain, including any condition or disorder associated therewith.

In another aspect, the invention provides a compound as defined herein above and below for use in the management of pain, including any condition or disorder associated therewith.

The invention provides a method of treating or managing pain and pain related disorders and symptoms in a subject in need thereof, said method comprising administering to a patient a composition as defined herein above and below.

The term “straight or branched C₁-C₁₀ alkyl” refers to a saturated hydrocarbon chain having 1, 2, 3, 4, 5, 6, 7, 8, 9 or 10 carbon atoms and the corresponding hydrogen atoms.

The term “straight or branched C₂-C₁₀ alkenyl” refers to an unsaturated hydrocarbon chain having 2, 3, 4, 5, 6, 7, 8, 9 or 10 carbon atoms and the corresponding hydrogen atoms and at least one double bond at any position in the chain.

The term “straight or branched C₂-C₁₀ alkynyl” refers to an unsaturated hydrocarbon chain having 2, 3, 4, 5, 6, 7, 8, 9 or 10 carbon atoms and the corresponding hydrogen atoms and at least one triple bond at any position in the chain.

The term aryl refers to an aromatic hydrocarbon single ring or fused ring system having between 6 to 18 carbon atoms. This term may also include a heteroaryl ring having at least one heteroatom.

The term “alkoxy” refers to a —OR radical wherein R is selected from a straight or branched C₁-C₁₀ alkyl, straight or branched C₂-C₁₀ alkenyl, straight or branched C₂-C₁₀ alkenyl.

The term “amine” refers to a —NRR′R″ radical wherein each of R, R′, R″ is independently selected from H, a straight or branched C₁-C₁₀ alkyl, straight or branched C₂-C₁₀ alkenyl, straight or branched C₂-C₁₀ alkenyl.

The term “amide” refers to a —C(═O)NRR′R″ radical wherein each of R, R′, R″ is independently selected from H, a straight or branched C₁-C₁₀ alkyl, straight or branched C₂-C₁₀ alkenyl, straight or branched C₂-C₁₀ alkenyl.

The term “halogen” refers to any one of F, Cl, Br, I.

The term “heteroatom” refers to any one of O, S, P, N.

When referring to Rings A, B and C being saturated, this should be understood to refer to said ring, independently, having only sigma bonds between the atoms forming the ring. When referring to Rings A, B and C being unsaturated, this should be understood to refer to said ring, independently, having at least one unsaturated bond (double and/or triple) at any position in the ring between the atoms forming the ring.

In some embodiments Ring A is a saturated 5, 6, 7, or 8 member ring (thus the ring consists of 5, 6, 7 or 8 atoms connected to each other with saturated single bonds only). In other embodiments, Ring A is an unsaturated 5, 6, 7 or 8 member ring (thus the ring comprises at least one unsaturated bond within the ring structure. Said unsaturated bond can be a double and/or a triple bond between any two atoms in the ring). In further embodiments Ring A is a 5-7 member ring having at least one heteroatom (thus said ring comprises at least one atom that is different than carbon being selected from O, N or S at any position in the ring. When valency permits heteroatom is substituted with one or more H, straight or branched C₁-C₁₀ alkyl, straight or branched C₂-C₁₀ alkenyl, straight or branched C₂-C₁₀ alkynyl.

The term “—C₍ₙ₎—” as used herein refers to a straight or branched hydrocarbon chain that can be saturated (i.e. having only single bonds connecting the atoms in the chain) or unsaturated (i.e. having at least one unsaturated bond, double or triple bond, connecting the atoms in the ring), having m carbon atoms. “—C₍ₙ₎—” chain can be optionally interrupted by at least one heteroatom, thus any two carbon atoms in the chain can be interrupted with at least one heteroatom between them (for example . . . —C—N—C— . . . ). Said heteroatom selected from O, N, S, P, when valency permits heteroatom is substituted with one or more H, straight or branched C₁-C₁₀ alkyl, straight or branched C₂-C₁₀ alkenyl, straight or branched C₂-C₁₀ alkynyl.

The term “—C₍ₘ₎—” as used herein refers to a straight or branched hydrocarbon chain that can be saturated (i.e. having only single bonds connecting the atoms in the chain) or unsaturated (i.e. having at least one unsaturated bond, double or triple bond, connecting the atoms in the ring), having m carbon atoms. “—C₍ₘ₎—” chain can be optionally interrupted by at least one heteroatom, thus any two carbon atoms in the chain can be interrupted with at least one heteroatom between them (for example . . . —C—N—C— . . . ). Said heteroatom selected from O, N, S, P, when valency permits heteroatom is substituted with one or more H, straight or branched C₁-C₁₀ alkyl, straight or branched C₂-C₁₀ alkenyl, straight or branched C₂-C₁₀ alkynyl.

In some embodiments, —C₍ₘ₎— is selected from a C₁-C₁₀ straight or branched alkylene, C₂-C₁₀ straight or branched alkenylene, C₂-C₁₀ straight or branched alkynylene. In some further embodiments, —C₍ₘ₎— is a C₁-C₁₀ straight or branched alkylene.

The compounds of the present application may include one or more asymmetric chiral center. Thus, the disclosure of the present application relates to any stereoisomer of the compound as it may occur. The chiral/assymetric centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “pain” as used herein should be understood to relate to any type of pain of any magnitude or duration, caused by any means (internal or external to the human body of a subject treated with a composition of the invention). For example, said pain may be caused by a bodily system whose dysfunction may be causing the pain (e.g., nervous, gastrointestinal). Said pain can be chronic pain or single episode pain, having any duration or pattern of occurrence. Said pain may be localized either in a single or multiple region of the body. Said pain may be of any intensity and time since onset. The pain treated by a composition or compound of the invention may be selected from at least one of the following classifications: nociceptive pain, inflammatory pain (typically associated with tissue damage and the infiltration of immune cells), pathological pain (typically associated with a disease state caused by damage to the nervous system or by its abnormal function such as fibromyalgia, irritable bowel syndrome, tension type headache, etc.).

The term “treatment of pain” as used herein refers to the administering of a therapeutic amount of a composition of the present invention comprising a compound of the present invention, which is effective to reduce, prevent or ameliorate pain of any magnitude felt by a subject including any undesired symptoms associated with the sensation of pain caused by any means (internal or external to the human body of a subject in need thereof).

The term “management of pain” as used herein refers to the administering of a therapeutic amount of a composition of the present invention comprising a compound of the present invention, which is effective to allow a subject suffering from any type or magnitude of pain, including any disorders or symptoms associated therewith, to control and ease the suffering and improve the quality of life of said subject, suffering from pain (including chronic pain).

The “effective amount” for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

The pharmaceutical compositions of the invention may comprise additionally any other suitable substances such as other therapeutically useful substances, diagnostically useful substances, pharmaceutically acceptable carriers or the like.

When referring to “composition(s)” or “pharmaceutical composition(s)” the present invention seeks to include any compositions suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents. Pharmaceutical compositions suitable for oral administration may be presented as discrete dosage units such as pills, tablets, drag-es or capsules, or as a powder or granules, or as a solution or suspension. The active ingredient may also be presented as a bolus or paste. The compositions can further be processed into a suppository or enema for rectal administration. The invention further includes a pharmaceutical composition, as hereinbefore described, in combination with packaging material, including instructions for the use of the composition for a use as hereinbefore described. For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any integer or step or group of integers and steps.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the structural characterization and categorization of known TRPV1 agonists and antagonists binding through the vanilloid-binding site (VBS).

FIGS. 2A-2E shows the tricyclic spirolactones: general architecture, functionality, topological sets, and synthetic protocol. Compounds illustrated in (2C) and (2D) were designed according to the reported methodology (2E) and were grouped according to the scaffold topology.

FIGS. 3A-3C show the specific tricyclic spirolactone topology is required for TRPV1 activation through the vanilloid-binding site. (3A) Top: Captured pseudocolor images of fura-2-loaded HEK293T cells transiently expressing rTRPV1, before (“Basal”) and after the application of synthesized tricyclic spirolactones (300 μM; ‘Comp.’), followed by the subsequent application of capsaicin (2 μM; “Cap”). Scale bar indicates level of intracellular calcium. Bottom: Box and whiskers plot showing the indicated compound (300 μM)-evoked calcium response of HEK293T cells expressing rTRPV1, normalized to the capsaicin (2 μM)-evoked response. Boxes represent the mean of 3-4 independent experiments (each n≥50 cells); only capsaicin-positive cells were analyzed. Statistical significance between the different compounds' normalized responses is indicated as follows: *p≤0.05; ***p≤0.001; ns, no statistical significance (ANOVA followed by multiple comparison test). (3B) Top: Captured pseudocolor images of fura-2-loaded HEK293T cells transiently expressing the wt rTRPV1 (“rV1”; top), chicken TRPV1 (“cV1”; middle), or the VBS mutated receptor rTRPV1-Y511G (“YG”; bottom), before (“Basal”) and after the application of compound 13 (300 μM; “13”), which was followed by the subsequent application of 2-APB (300 μM; “2APB”). Scale bar indicates level of intracellular calcium. Bottom: changes with time of intracellular calcium levels in fura-2-loaded HEK293T expressing rat TRPV1 (“rTRPV1”; red line), rat TRPV1-Y511G (“rTRPV1(YG)”; purple line), and chicken TRPV1 (“cTRPV1”; orange line) in response to compound 13 (300 μM; “13”; light gray bar), which was followed by the subsequent application of 2-APB (300 μM; “2APB”; gray bar). Each trace represents an average of 75-130 cells sensitive to 2-APB. Note that both capsaicin-insensitive constructs (chicken and VBS-mutated rat TRPV1) were not activated by compound 13. (3C) Top: Captured pseudocolor images of fura-2-loaded HEK293T cells transiently expressing rTRPV1, before (“Basal”) and after the application of saturated tricyclic spirolactones (300 μM; “Comp.”), which was followed by the subsequent application of capsaicin (2 μM; “Cap”). Scale bar indicates the level of intracellular calcium. Bottom: Box and whiskers plot shows the indicated compound (300 μM)-evoked calcium response of HEK293T cells expressing rTRPV1, normalized to the capsaicin (2 μM)-evoked response. Boxes represent the mean of 3-4 independent experiments (each n≥50 cells); only capsaicin positive cells were analyzed. Statistical significance between the different compounds' normalized responses is indicated as follows: **p≤0.01; ***p≤0.001; ns, no statistical significance (ANOVA followed by multiple comparison test).

FIG. 4 shows the specific tricyclic spirolactone topology is required for TRPV1 activation. Box and whiskers plot shows the indicated compound (300 μM)-evoked calcium response of HEK293T cells expressing rTRPV1, normalized to the capsaicin (2 μM)-evoked response. Boxes represent the mean of 3-4 independent experiments (each n≥50 cells); only capsaicin positive cells were analyzed. Statistical significance between the different active compounds' normalized responses is indicated as follows: ***p≤0.001; ns, no statistical significance (ANOVA followed by multiple comparison test).

FIGS. 5A-5B shows the results of attaching aromatic anchors to tricyclic spirolactones dramatically increases TRPV1 response. Captured pseudocolor images of fura-2-loaded HEK293T cells transiently expressing rat TRPV1, before (“Basal”) and after the application of aromatically anchored tricyclic spirolactones (300 μM; “Comp.”), which was followed by the subsequent application of capsaicin (2 μM; “Cap”). Scale bar indicates the level of intracellular calcium. Bottom: Box and whiskers plot shows the indicated compound (300 μM)-evoked calcium response of HEK293T cells expressing rTRPV1, normalized to the capsaicin (2 μM)-evoked response. Boxes represent the mean of 3-4 independent experiments (each n≥50 cells); only capsaicin positive cells were analyzed. Statistical significance between the different compounds normalized responses is indicated as follows: ***p≤0.001; ns, no statistical significance (ANOVA followed by multiple comparison test). Note that the strongest response was evoked by the integration of the aromatic anchors (of benzylic or vanilloid nature; compounds 31 and 32) within the tricyclic scaffold of compound 13.

FIGS. 6A-6C show the results indicating that compounds 13 and 32 evoke a robust outwardly rectifying channel activation. (6A) Current-voltage relationship traces in response to compound 32 (1 mM; “32”; blue trace) and capsaicin (2 μM; “Cap”; red trace) in HEK293T cells transiently expressing rTRPV1. Note that both capsaicin and compound 32 elicited robust, outwardly rectifying currents of similar magnitude. (6B) Normalized concentration-response relationships for 13 (cyan line) and 32 (blue line) of rTRPV1 stably expressed in HEK293 cells. Each point represents the average (±SEM) response of 50 cells. Solid lines are fit to the Hill equation with EC50 and n for 13 of 127.0±8.9 μM and 2.7 and for 32 of 12.8±1.6 μM and 1.4, respectively. (6C) Top: Captured pseudocolor images (left) and changes with time (right) of fura-2-loaded HEK293T cells stably expressing the wt rTRPV1 in the presence of capsazepine (“CPZ”;20 μM) before (“Basal”) and after two application of compound 32 (100 μM; “32a” and 300 μM; “32b”), which was followed by the subsequent application of capsaicin (3 μM; ‘Cap’). The trace represents an average of 165 cells sensitive to capsaicin. Bottom: Captured pseudocolor images (left) and changes with time (right) of fura-2-loaded HEK293T cells stably expressing the wt rTRPV1 before (“Basal”) and after application of compound 32 (300 μM; “32b”), which was followed by the immediate application of capsazepine (“CPZ”;20 μM). Lastly, a subsequent application of capsaicin (3 μM; “Cap”) was administrated. The trace represents an average of 140 cells sensitive to capsaicin. Scale bar indicates level of intracellular calcium.

FIGS. 7A-7B show that the aromatically anchored saturated tricyclic spirolactone evoke channel activation. (7A) Box and whiskers plot shows the indicated compound-evoked calcium response of HEK293T cells expressing rTRPV1, normalized to the capsaicin (2 μM)-evoked response. Compound 13 (1 mM) was added alone (white bar), and following application of compound 18 at 0.3 mM (light gray) or 1 mM (dark gray). Boxes represent the mean of two independent experiments (each n≥50 cells); only capsaicin positive cells were analyzed. Statistical significance is indicated as follows: ***p≤0.001; ns, no statistical significance (ANOVA followed by multiple comparison test). (7B) Top: captured pseudocolor images of fura-2-loaded HEK293T cells transiently expressing rTRPV1, before (“Basal”) and after the application of compound 34 (300 μM; “#34”), followed by the subsequent application of capsaicin (2 μM; “Cap”). Scale bar indicates level of intracellular calcium. Bottom: changes with time of intracellular calcium levels as described above (n=50 cells).

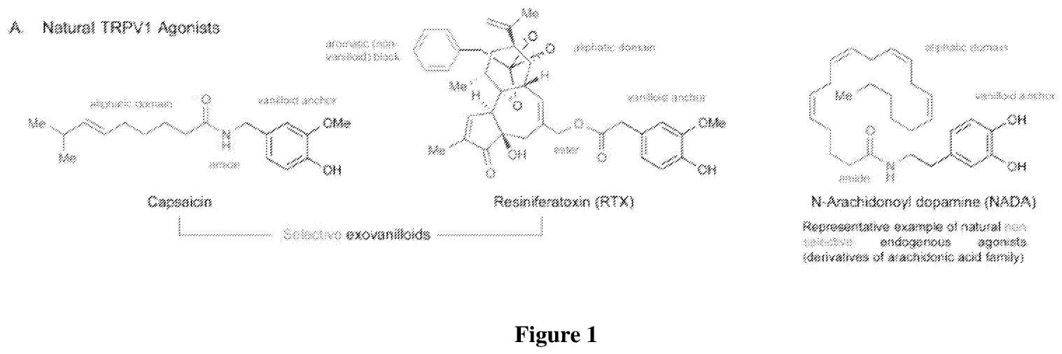

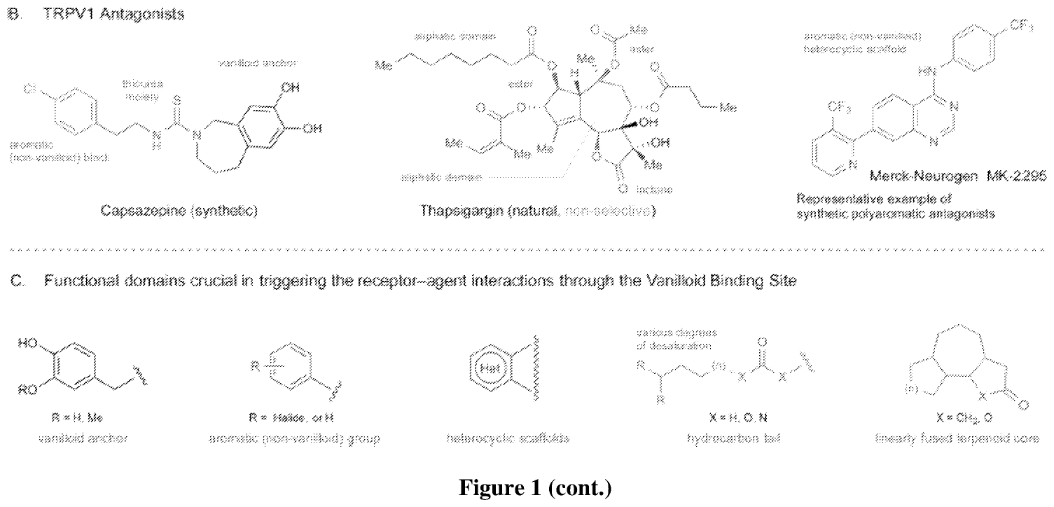

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

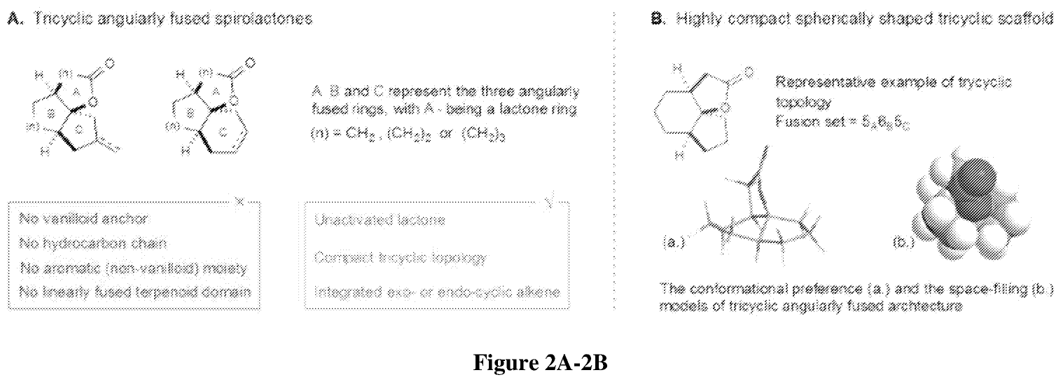

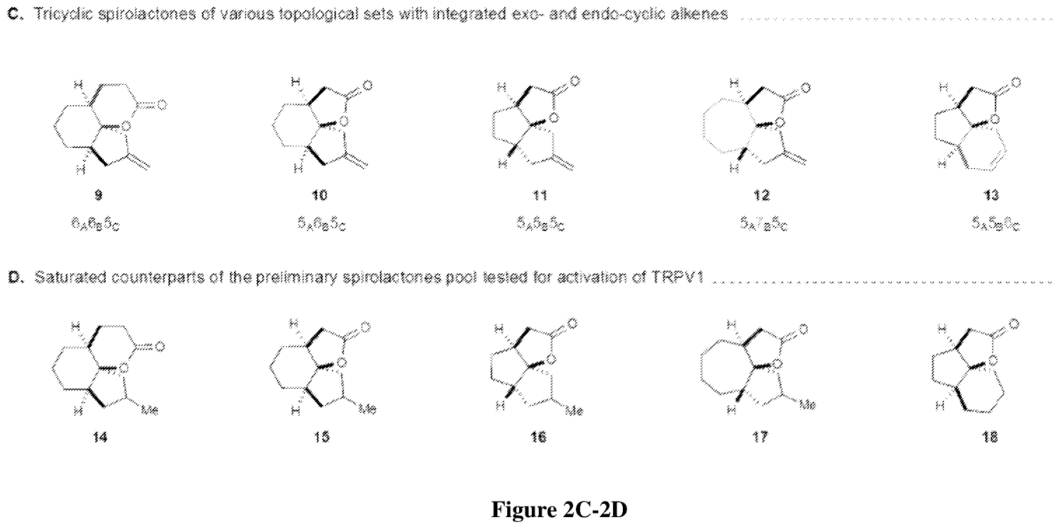

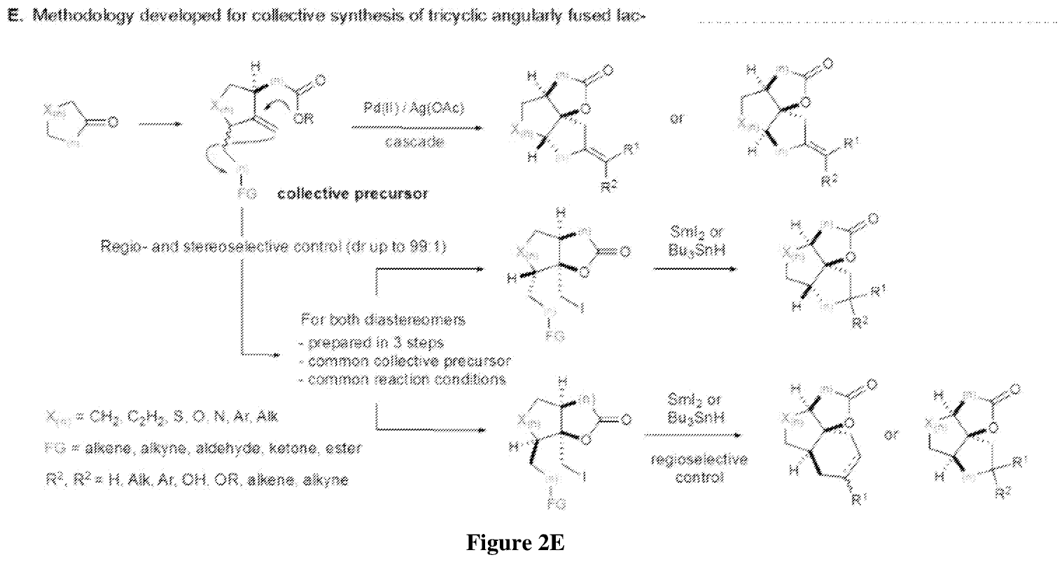

Tricyclic spirolactones are frequently observed as scaffold segments of various natural biochemical compounds. Examples of these structures have been identified in carbohydrates, terpenoids, antibiotics, and many other compounds. Recently, we reported general and collective syntheses of phylogenetically different tricyclic, angularly fused spirolactones via controlled cyclizations of easily accessible common cycloalkylmethylene key precursors (FIG. 2E). We operated under the assumption that novel synthetic scaffolds, which are small, rigid, and highly reminiscent of natural scaffolds, could serve as operational ligands for TRPV1. Many spiranoid lactones have been firmly established to demonstrate pharmacological activity. Thus, we were motivated to apply our designed architectures to the TRPV1 model.

Tricyclic spirolactones activate TRPV1 through the VBS domain. Following the established protocol, we prepared a series of angularly fused lactones featuring various tricyclic topologies (displaying varying ring sizes and fusion combinations) and diverse electronic and steric characteristics (FIG. 2C). Venturing beyond existing conceptions, we decided to experiment with molecules that do not have integrated vanilloid domains or aromatic rings. The only key functionality retained within the tested tricyclic scaffolds was exo- or endo-integrated single alkene element (another frequently observed functional group for known exo/endogenous TRPV1 agonists).

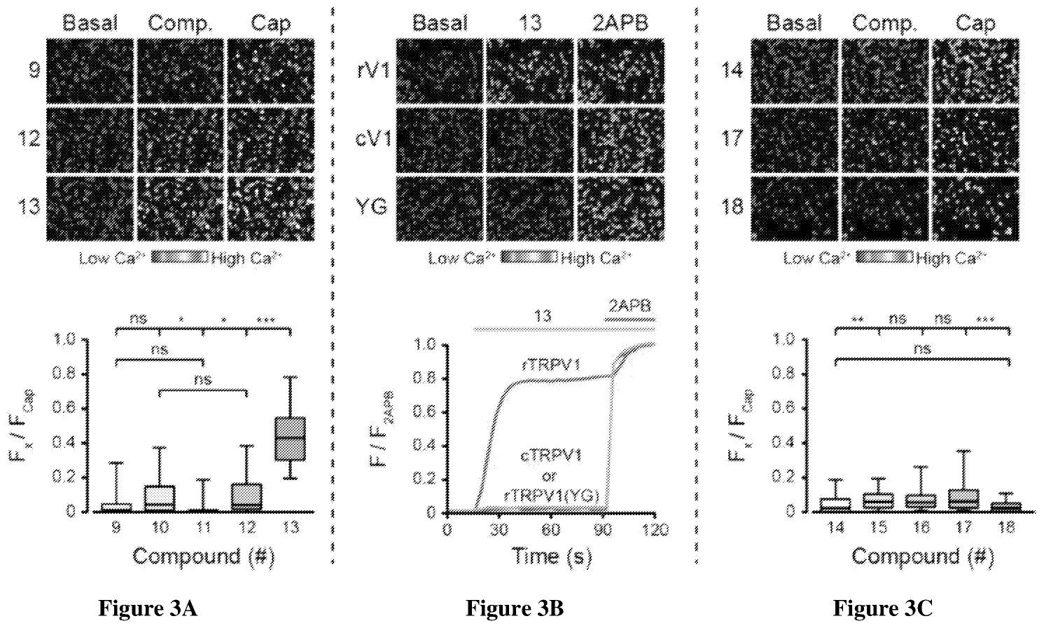

To examine whether the compact tricyclic spirolactones activate TRPV1, we scanned a library of synthesized compounds illustrated in FIG. 2C. Taking advantage of the high permeability to calcium displayed by TRPV1, we examined the activation profile of our library using live-cell calcium imaging of HEK293T cells transiently expressing rat TRPV1 (rTRPV1). Although our compounds are less hydrophobic than other VBS-binding molecules (e.g., capsaicin), most synthesized compounds could not be prepared in physiological salt solutions (i.e., Ringer) at concentrations greater than 1 mM without adding cytotoxic levels of DMSO. To avoid any nonspecific cellular activation, we did not exceed 0.1% of DMSO in the final solutions. As shown in FIG. 3A, all compounds activated capsaicin-sensitive cells at 0.3 mM; however, different activation levels were observed. Interestingly, compound 13 demonstrated an unexpectedly robust response compared to the other compounds (FIG. 3). To confirm that the observed calcium increase is a direct result of TRPV1 activation through the VBS, we used chicken TRPV1, which is insensitive to vanilloids28, and VBS-mutated rat TRPV1 (rTRPV1(Y511G)). As shown in FIG. 3B, compound 13 did not activate both vanilloid-insensitive channels. The functionality and expression of these channels were verified by their sensitivity to 2-aminoethoxydiphenyl borate (2-APB), a non-VBS TRPV1 activator. We next investigated the selectivity of compound 13 to TRPV1 by examining the neuronal response of acutely dissociated trigeminal ganglia neurons. Our results indicate that compound 13 activated only capsaicin sensitive neurons. A possible explanation to the observed lower response of 13 in comparison to capsaicin might be the accessibility of the compound to the intracellular VBS. To test this possibility, we recoded the response of rTRPV1 transiently expressed in HEK293T cells in the whole-cell or inside-out patch clamp configurations. Our data suggest that similarly to capsaicin, compound 13 evoke similar channel responses from both sides of the membrane. Thus, we found that compact, functionally inactive, tricyclic spirolactones could specifically activate TRPV1 through its VBS without any of the structural elements previously thought to be necessary.

TRPV1 activation by tricyclic spirolactones depends on their saturation and the functionality state. After concluding that unsaturated structures can activate the TRPV1 via the VBS, we were compelled to examine the functionality of fully saturated variants (i.e., the same range of molecules without alkenes, as shown in FIG. 2B). Compounds 14-18 were designed according to the reported methodology and tested at 0.3 mM using live-cell calcium imaging of HEK293T cells transiently expressing rTRPV1. As shown in FIG. 3C, the saturation of these compounds decreased their ability to activate TRPV1. Interestingly, the most dramatic decrease in the level of activation was detected for compound 18, the saturated analogue of 13.

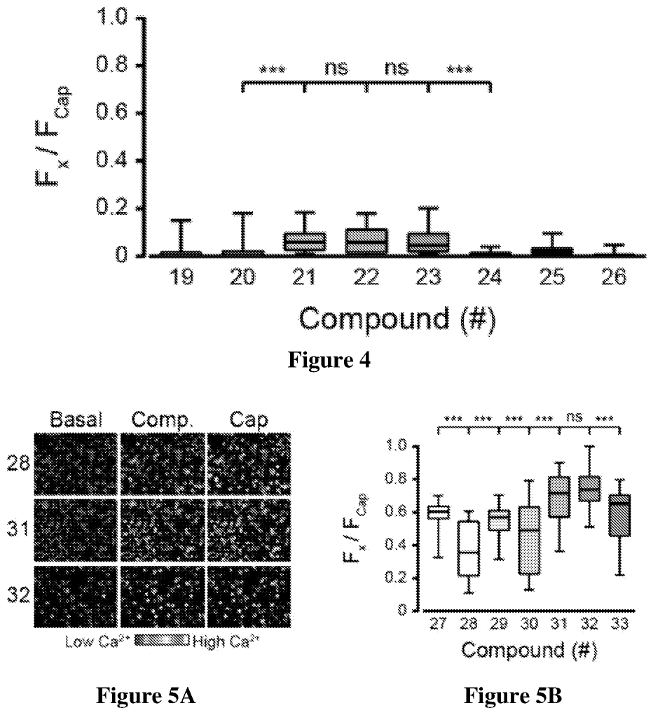

We were intrigued by the possibility of applying the established protocol to other spirolactones. We thus prepared a range of tricyclic scaffolds sharing various topologies and functional groups integrated within the scaffold, as shown in FIG. 4. Again, attention was focused on the scope of the substrate-receptor interaction with respect to capsaicin. For the series of compounds 19-22, the alkene group was substituted by a hydroxy residue, and no significant activity was detected.

This observation strengthens our hypothesis that an alkene element is indispensable for initiating the substrate-receptor interaction. However, for compounds 24-26, which resemble the topology of compounds 10 and 15 (active compounds, see FIG. 3) and retain the double bond, an introduction of heteroatoms (O, S) within the central ring significantly reduced potency. Similarly, no activity was detected for a substrate with a substituted alkene functional group (23, FIG. 4).

Integration of the aromatic anchor across the active scaffolds. Previous studies have clearly indicated the importance of the aromatic domains in TRPV1 activation through the VBS. Thus, we postulated that the introduction of aromatic domains would provide additional tools to manipulate and better understand our system. To test the changes in the intensity of binding (receptor-ligand interaction), the leading topologically diverse scaffolds of compounds 10, 12, and 13 (FIG. 2) were combined with aromatic anchors (benzyl and vanilloid groups) to generate advanced tricyclic platforms (FIG. 5). The OH-bearing compounds 21 and 22 (FIG. 4) were modified by the installation of aromatic groups at different sites (compounds 27-29 were designed according to reported methodology)22. In a similar fashion, compounds 13, 15, and 17 (FIG. 2) were anchored with benzyl and vanilloid groups to generate architectures 30-33.

To examine their activation profile, the prepared compounds (FIG. 5A) were analyzed using live cell calcium imaging (as described above, FIG. 3). All compounds displayed a significant increase in activity compared to their parent compounds. These results indicate that the aromatic anchor increases the level of agonist activation, but it is not a crucial functional element.

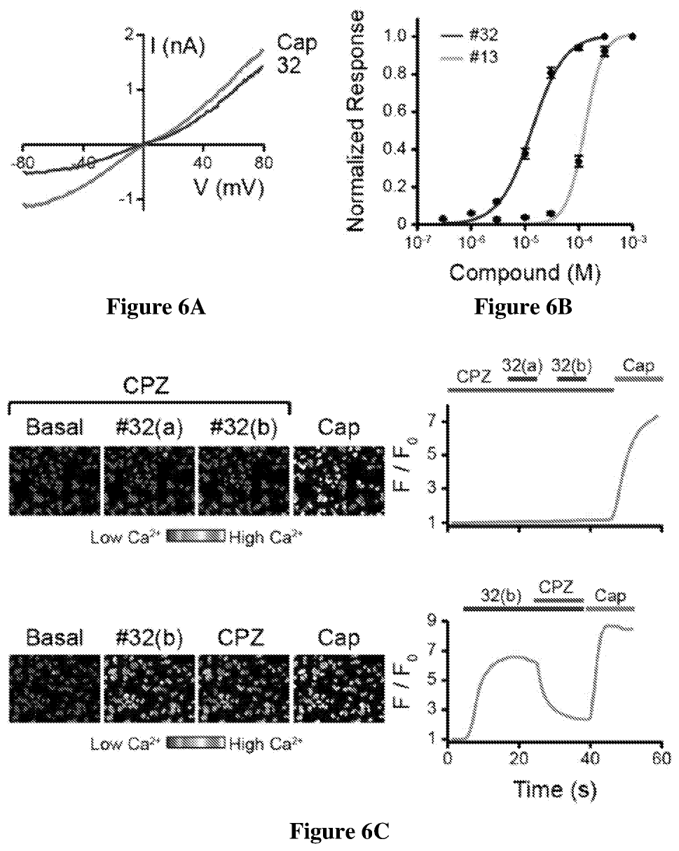

Tricyclic spirolactones evoke robust channel activation. TRPV1 is a non-selective cationic ion channel with a typical current profile. To examine whether our novel structure architecture activates the channel similarly as other VBS -associated agonists, we analyzed the current profile using the whole-cell configuration of the patch clamp technique. Using voltage ramps between −80 and +80 mV, we analyzed the channel response to 32 and capsaicin. As shown in FIG. 6A, compound 32 elicits the typical TRPV1 outwardly rectifying current, which is similar to capsaicin.

To further analyze the role of the aromatic anchor in TRPV1 activation by spirolactones, we compared the affinity of 13 to its vanilloid derivative 32. As clearly showed in FIG. 6B, a one order of magnitude difference was obtained when the vanilloid moiety was attached to 13 (13: EC50=127.0±8.9 μM; 32: EC50=12.8±1.6 μM). Moreover, the Hill coefficient was attenuated as well (13: n=2.7±0.5; 32: n=1.4±0.2), indicating a complex activation mechanism of spirolactones without aromatic moiety (FIG. 6B). Interestingly, the Hill coefficient of 32 highly resemble the previous reported Hill coefficients of capsaicin.31 Lastly, we verified that the VBS competitive inhibitor, capsazepine (CPZ), inhibits the robust activation of TRPV1 by 32. As shown in FIG. 6C, pre-incubation with CPZ or application CPZ following pre-activation by 32 resulted in channel inhibition, pointing to the specificity of TRPV1 activation by spirolactones and their derivatives.

Thus, like other vanilloids, our novel scaffold can robustly activate TRPV1 through the VBS. On the basis of the obtained results, we were intrigued to examine the behavior of fully saturated spirolactones that were incorporated with aromatic modules. Taking into account the fact that TRPV1 activation by tricyclic spirolactones depends on their saturation, we were interested if such structural modification (compensation of the alkene group with aromatic moiety) could cause the activation of the receptor.

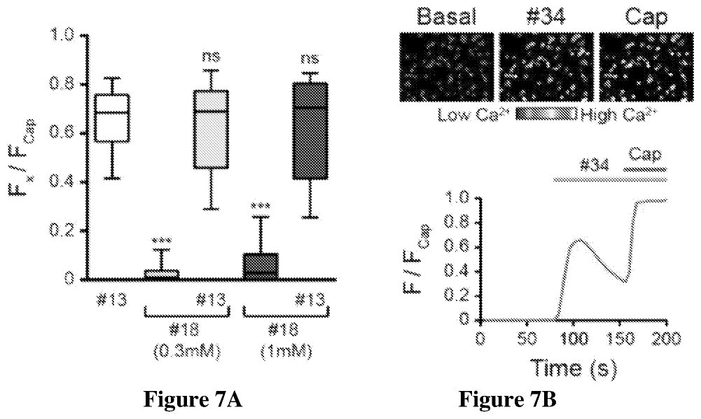

To test this assumption, we conducted an experiment in which compound 18 (saturated analogue of active 13; FIG. 2) was integrated with benzyl group to generate structural hybrid 34 (FIG. 7A). First, we tested the binding interaction of compound 18 in the VBS by analyzing its inhibitory effect on 13. As shown in FIG. 7B, spirolactone 18 had no effect on the response of it's unsaturated analogue 13. However, the resulted spino-derivative 34 was evoke significant channel activation (FIG. 7C). Nevertheless, compound 34 demonstrate a transiently response in comparison to compound 13 or 32 (compare FIG. 7C to FIGS. 3B and 6C). We address the ability of fully saturated-benzylated lactone 34 to transiently activate TRPV1, to the prolonged duration of the molecule in the VBS due to the aromatic interactions between the benzyl group and the VBS. However, its saturated structure does not enable it to evoke stable channel opening.

In conclusion, the overarching value obtained from our inquiry is the ability to advance the existing TRPV1 activation model, which is supported by over two decades of dedicated research. Three molecular pharmacophores/domains/functional groups—the vanilloid, benzene, or other aromatic heterocycle; the lipophilic hydrocarbon chain; and the linearly fused terpenoid moiety—have been thought indispensable for an agonist/antagonist to trigger receptor-agent interactions through the VBS. This understanding was achieved because the interactive potency of the residual structure is either lost or significantly diminished when these key elements are removed. Herein, we establish the existence of a conceptually unique family of activating agents that interface with the TRPV1 receptor through the VBS. The reported series of molecules-highly compact, tricyclic, spherical spirolactones-contain none of the structural domains previously believed to be integral for receptor interaction.

When these lactones were anchored with an aromatic residue, the efficacy of the compound increased. However, based on our observations, the nature of the aromatic anchor is inconsequential; similar efficacy was detected for the basic benzylic-enriched scaffolds and their vanilloid alternatives. As previously demonstrated,8,14-18 the aromatic interaction between agonist and the tyrosine residue in the VBS augments the TRPV1 response. This analysis led to the assumption that this interaction is not required for the binding to result in TRPV1 activation. Here, we show that TRPV1 activation through the VBS does not depend on the aromatic interaction. As evidenced by the robust response of compound 13 compared to other spirolactones, we believe that other yet-to-be-determined interactions are necessary for the activation of TRPV1 by hydrophobic molecules. The addition of an aromatic moiety to compound 13 created compounds (31 and 32) with capsaicin-like activity. Thus, our compounds reveal the flexibility of the VBS.

Further structural analysis is required to understand the interactions that control the activation of TRPV1 through this binding pocket.

We humbly hope that our findings uncover new potential in the area of pain treatment, providing a novel synthetic platform for further research. Our synthetic strategy is short, regioselective, and offers the possibility to access a broad spectrum of quaternary carbon-centered spiranoid scaffolds.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound of general formula (I): 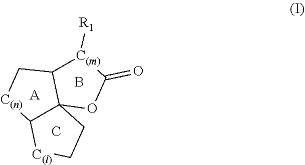
wherein
R₁ is selected from straight or branched C₁-C₁₀ alkyl, straight or branched C₂-C₁₀ alkenyl, straight or branched C₂-C₁₀ alkynyl; each optionally interrupted by at least one heteroatom and substituted by at least one aryl group optionally substituted with at least one of —CN, —OH, alkoxy, amine, amide and halogen;
Rings A, B and C are each optionally a saturated or unsaturated ring having optionally at least one heteroatom; and are each optionally substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;
R₄, R₅, R₆, R₇ are each independently selected from H, halogen, —OH, —NH₂, straight or branched C₁-C₁₀ alkyl, straight or branched C₂-C₁₀ alkenyl, straight or branched C₂-C₁₀ alkynyl, aryl, —O(C₁-C₁₀)alkyl, NH₂, amine; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;
n is an integer selected from 1-10;
—C₍ₙ₎— is selected from a straight or branched alkylene, straight or branched alkenylene, straight or branched alkynylene; optionally interrupted by at least one heteroatom;
m is an integer selected from 1-10;
—C₍ₘ₎— is selected from a straight or branched alkylene, straight or branched alkenylene, straight or branched alkynylene; optionally interrupted by at least one heteroatom;
l is an integer selected from 2-10;
—C₍ₗ₎— is selected from a straight or branched alkylene, straight or branched alkenylene, straight or branched alkynylene; optionally interrupted by at least one heteroatom.

2. A compound of general formula (II): 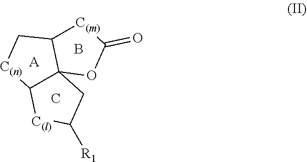
wherein
R₁ is selected from straight or branched C₁-C₁₀ alkyl, straight or branched C₂-C₁₀ alkenyl, straight or branched C₂-C₁₀ alkynyl; each optionally interrupted by at least one heteroatom and optionally substituted by at least one aryl group optionally substituted with at least one of —CN, —OH, alkoxy, amine, amide, halogen;
Rings A, B and C are each optionally a saturated or unsaturated ring having optionally at least one heteroatom; and are each optionally substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;
R₄, R₅, R₆, R₇ are each independently selected from H, halogen, —OH, —NH₂, straight or branched C₁-C₁₀ alkyl, straight or branched C₂-C₁₀ alkenyl, straight or branched C₂-C₁₀ alkynyl, aryl, —O(C₁-C₁₀)alkyl, NH₂, amine; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl;
n is an integer selected from 1-10;
—C₍ₙ₎— is selected from a straight or branched alkylene, straight or branched alkenylene, straight or branched alkynylene; optionally interrupted by at least one heteroatom;
m is an integer selected from 1-10;
—C₍ₘ₎— is selected from a straight or branched alkylene, straight or branched alkenylene, straight or branched alkynylene; optionally interrupted by at least one heteroatom;
l is an integer selected from 2-10;
—C₍ₗ₎— is selected from a straight or branched alkylene, straight or branched alkenylene, straight or branched alkynylene; optionally interrupted by at least one heteroatom.

3. A compound according to claim 1 or 2, wherein R₁ is straight or branched C₁-C₁₀ alkyl; substituted by at least one aryl group optionally substituted with at least one of —CN, —OH, alkoxy, amine, amide, halogen.

4. A compound according to claim 1 or 2, wherein R₁ is straight or branched C₂-C₁₀ alkenyl; substituted by at least one aryl group optionally substituted with at least one of —CN, —OH, alkoxy, amine, amide, halogen.

5. A compound according to claim 1 or 2, wherein R₁ is selected from straight or branched C₁-C₁₀ alkyl, straight or branched C₂-C₁₀ alkenyl that is interrupted by at least one heteroatom; substituted by at least one aryl group optionally substituted with at least one of —CN, —OH, alkoxy, amine, amide, halogen.

6. A compound according to claim 1 or 2, wherein R₁ is selected from straight or branched C₁-C₁₀ alkyl, straight or branched C₂-C₁₀ alkenyl substituted by at least one phenyl optionally substituted with at least one of —CN, —OH, alkoxy, amine, amide and halogen.

7. A compound according to claim 1 or 2, wherein R₁ is selected from straight or branched C₁-C₁₀ alkyl, straight or branched C₂-C₁₀ alkenyl substituted by at least one phenyl substituted with at least one of —CN, —OH, alkoxy, amine, amide and halogen.

8. A compound according to claim 1 or 2, wherein Ring C is a saturated ring.

9. A compound according to claim 1 or 2, wherein Ring C is a saturated ring substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl.

10. A compound according to claim 1 or 2, wherein Ring C is an unsaturated ring.

11. A compound according to claim 1 or 2, wherein Ring C is an unsaturated ring substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen; each of said straight or branched C₁-C₁₀ alkyl, straight or branched C₂-C₁₀ alkenyl, straight or branched C₂-C₁₀ alkynyl are optionally substituted with at least one of —CN, —OH, alkoxy, amine, amide, halogen, aryl.

12. A compound according to claim 1 or 2, wherein m=1, —C(m)- being a C1-alkylene optionally interrupted by at least one heteroatom.

13. A compound according to claim 1 or 2, wherein n=1, —C(n)- being C1-alkylene, optionally interrupted by at least one heteroatom.

14. A compound according to claim 1 or 2, wherein n=2, —C(n)- being C₂-alkylene, optionally interrupted by at least one heteroatom.

15. A compound according to claim 1 or 2, wherein n=3, —C(n)- being C₃-alkylene, optionally interrupted by at least one heteroatom.

16. A compound according to claim 1 or 2, wherein l=1, —C(l)- is a C1-alkylene, optionally interrupted by at least one heteroatom.

17. A compound according to claim 1 or 2, wherein l=2, —C(l)- is a C₂-alkenylene, optionally interrupted by at least one heteroatom.

18. A compound according to claim 1, being selected from:

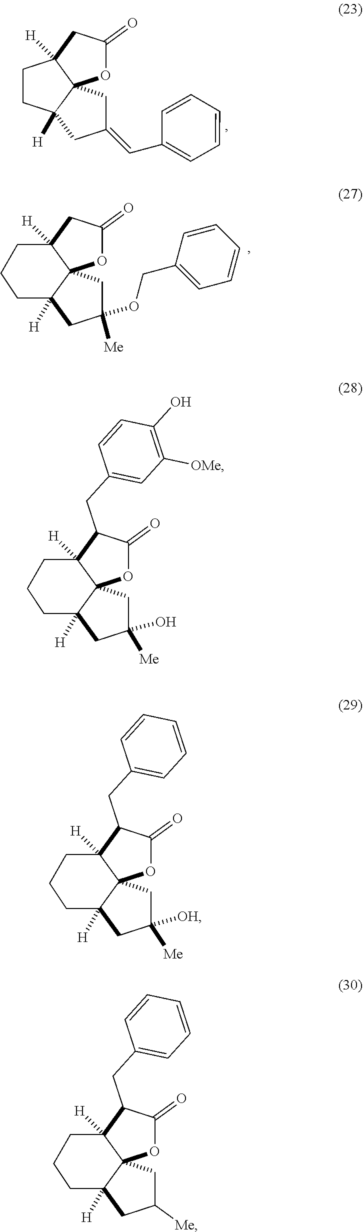

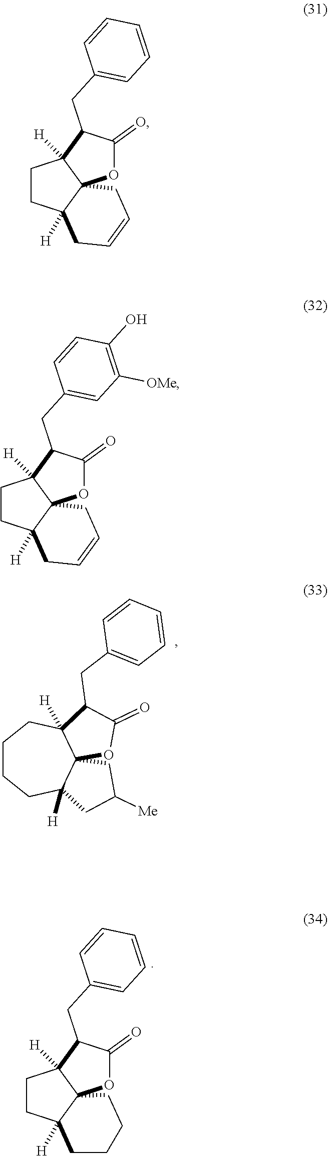

19. A pharmaceutical composition comprising at least one compound according to claim 1 or 2.

20. (canceled)

21. (canceled)

22. A method of treating or managing pain and pain related disorders and symptoms in a subject in need thereof, said method comprising administering to a patient a composition of claim 19.
